(19) United States
(12) Patent Application Publication
(10) Pub. No.: US 2008/0050441 A1
(43) Pub. Date: Feb. 28, 2008

(54) Process for the preparation of solid sterile active pharmaceutical ingredient

(76) Inventors: Enrico Bettetini, Novara (IT); Piercorrado Bordoni, Novara (IT); Francesca Cofano, Torino (IT); Alfio Messina, Lodi (LO) (IT); Massimiliano Palladino, Inveruno (MI) (IT)

Correspondence Address: KENYON & KENYON LLP, ONE BROADWAY, NEW YORK, NY, 10004, US

(21) Appl. No.: 11/880,286

(22) Filed: Jul. 20, 2007

Related U.S. Application Data
(60) Provisional application No. 60/832,349, filed on Jul. 20, 2006.
(60) Provisional application No. 60/847,289, filed on Sep. 25, 2006.

(51) Int. Cl. A61K 9/14; A61K 31/46; A61K 31/57; A61K 31/58

(52) U.S. Cl. 424/489; 514/174; 514/178; 514/180; 514/291

(57) ABSTRACT

The present invention provides a method of preparing a packed sterile solid active pharmaceutical ingredient, in particular sterile steroids such as a glucocorticosteroid acid.

### CROSS REFERENCE TO RELATED APPLICATIONS

The present application claims the benefit of the following U.S. Provisional Patent Application Nos. 60/832,349, filed Jul. 20, 2006 and 60/847,289, filed Sep. 25, 2006. The contents of these applications are incorporated herein by reference.

### FIELD OF THE INVENTION

The present Invention relates to a process for the production of a packaged micronized sterile solid active pharmaceutical ingredient, in particular sterile steroids.

### BACKGROUND OF THE INVENTION

Sterilization of the final filled container as a dosage form or of the final packaged device is considered as the best process for ensuring the minimal risk of microbial contamination (“Terminal Sterilization”, for example gamma irradiation and heat cycles). However, there is a substantial class of solid active pharmaceutical ingredients (APIs) that cannot be sterilized terminally, since all such sterilization techniques affect the quality of the product (for example gamma irradiation and heat cycles techniques frequently cause degradation of the API, and heat cycles in solid suspensions can affect polymorph type and particle size distribution of suspended API). Moreover, formulations involving solid API's (Sterile Suspensions for parenteral use, Sterile Suspensions for Respiratory use, Inhalation Powders, etc . . . ) cannot be sterilized by filtration, as most of the particles of API would be retained on the sterilizing filter. Moreover, there is a need for tight control of polymorphic form and particle size distribution in the finished dosage form since bioavailability and consequently therapeutical efficacy are strongly affected by the above parameters. Therefore, procedures for the preparation of sterile solid API, in particular steroids, were developed using a series of aseptic steps including filtration of sterile solutions and sterile micronization procedures.

International Patent Application Publication WO 99/25359 discloses the sterilization of a powder form of a glucocorticosteroid, sterile glucocorticosteroids and sterile formulations containing glucocorticosteroids. The sterilization is done by heating the steroid to a temperature of about 100° C. to about 130° C. using an oven or a flow of hot gas; however, the surface characteristics of the steroid crystal may be altered with this process.

International Patent Application Publication WO 99/25359 relates also to other methods used to sterilize solid glucocorticosteroid, however, it claims that these methods are not suitable for sterilization of steroids, or other sensitive APIs due to their sensitivity to temperature, and due to the strict limitations of the pharmacopoeia with respect to impurity content.

Hence, there is a need to develop a new process for the preparation of sterile solid API.

### SUMMARY OF THE INVENTION

A process to produce a packaged sterile solid active pharmaceutical ingredient (API) in a glove box or laminar air flow (LAF) hood comprising the steps of a) providing a solution of the API, b) filtering the solution; c) precipitating and recovering the API from the solution; d) micronizing the API; and e) packaging the API, wherein at least the steps d) and e) are carried out in a sterile glove box or LAF hood.

### BRIEF DESCRIPTION OF THE DRAWINGS

FIG. 1: illustrates the sterile production unit.

FIG. 2: illustrates the sterile production unit.

### DETAILED DESCRIPTION OF THE INVENTION

As used herein, the term “sterile” refers to the complete absence of viable micro-organisms. However, this absolute definition cannot practically be applied to an entire lot because to verify the complete absence of micro-organism all the material of the batch should be incubated, with complete destruction of every finished article. The sterility of a lot purported to be sterile is therefore defined in terms with respect to probabilities, where the likelihood of a contaminated unit or article is acceptably remote (10⁻⁶). Such a state of sterility assurance can be established only through the validation and use of adequate sterilization cycles and subsequent aseptic processing, under appropriate current good manufacturing practice, and not by reliance solely on sterility testing on a sample of limited quantity. As such, a sterile unit or article is defined as a unit in which, based on statistics related to the conditions of preparation and sterilization of that specific product and of that specific batch, less than one unit in a million of the product is exposed to the risk of not being sterile, i.e., the probability of finding a non-sterile unit (PNSU=Probability of Non Sterile Unit) must therefore be lower than 10⁻⁶.

The present invention relates to a process to produce a sterile solid API, in particularly, a high potentency API such as glucocorticosteroids, and its aseptic micronization, wherein the sterile solid API can be used directly for formulation. This process takes into consideration the protection of the operator and of the product by performing the sterilization under mild conditions, i.e., without heating, and its handling and micronization either in a laminar air flow (LAF) hood also referred to herein as a glove box or in a clean room. For this reason, the operator doesn't have to wear personal protective equipment to avoid the hazards of the process, and the sterile API obtained by this process has lower risk of microbial contamination and air degradation of the product.

An example of a steroid of the present invention is triamcinolone, a typical impurity for the steroids such steroid is the 21-aldehyde impurity thereof, 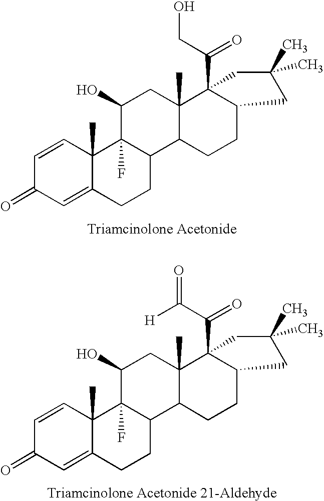
an impurity that is restricted by the pharmacopoeia, and is formed by a reaction of the steroid with oxygen, a reaction known also to be affected by heat. Moreover, the use of a glove box instead of a clean room simplifies the production by avoiding open transfer of the API from one vessel to another, as done in a clean room, while preserving the sterilization conditions. Also, this process can be scaled up easily and efficiently.

The present invention provides a process to produce a packaged micronized sterile solid API in a glove box or LAF hood comprising the steps of sterilizing by filtration a solution of the API; precipitating the API; recovering the API, discharging the API, and micronizing and packaging the API, wherein at least the step of discharging and the step of micronizing and packaging the API are carried out in a LAF hood or glove box. Preferably, all steps subsequent to filtering the solution of the API are carried out under aseptic conditions of which at least discharging the API and micronizing and packing the API are carried out in a glove box or LAF hood.

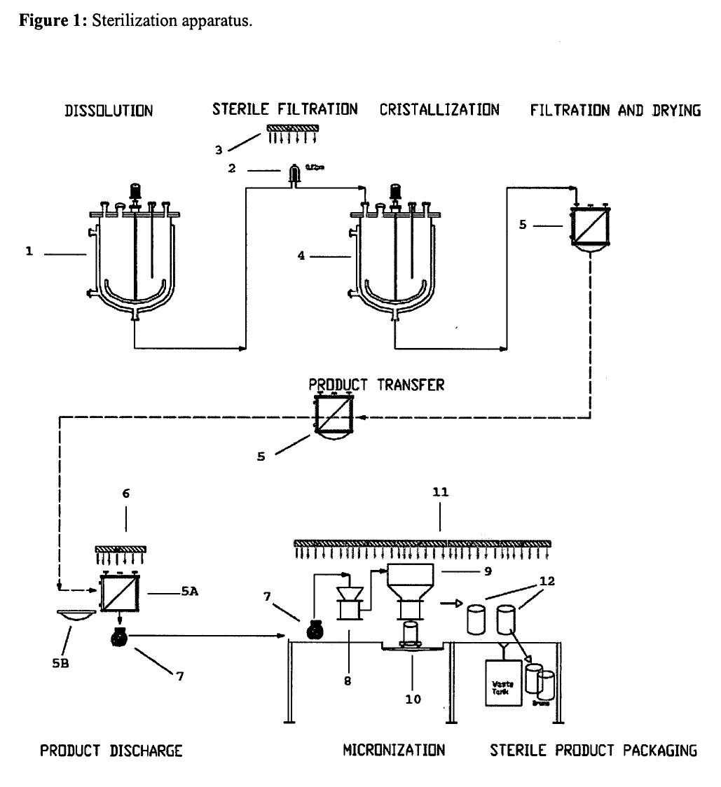

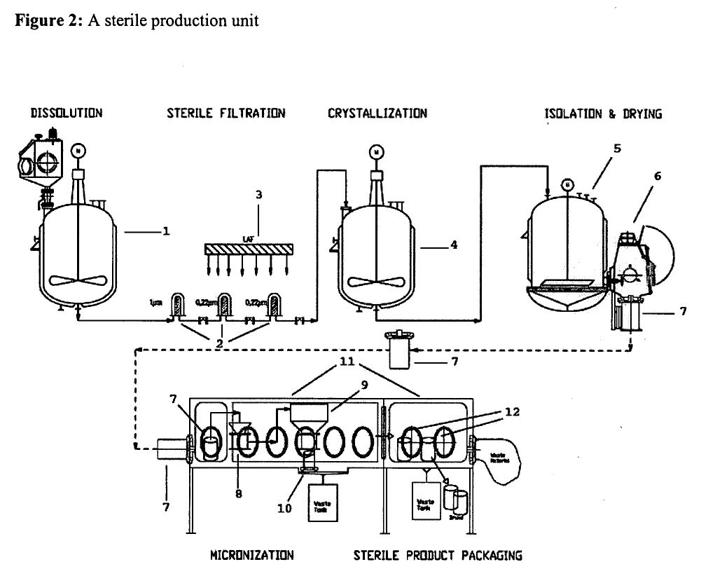

Preferably, the process is carried out in the apparatus presented in FIG. 1 or FIG. 2. A preferred embodiment of the invention will now be described in detail with reference to FIG. 1.

As set forth in FIG. 1, a solution of the API is prepared in a first reactor 1 by dissolving the API in a suitable solvent. Optionally, the solution may be heated. The solution is then sterilized by filtration over filter 2, preferably filter 2 is a 0.22 micron sterilization cartridge, to dispel microorganisms and other contaminating agents. Preferably, the filtration cartridge is mounted in a laminar air flow (LAF) hood or glove box 3. Alternatively, or additionally, other types of membrane filtration devices may be employed for filtration (e.g., filter disks or filter cartridges of varying sizes and micron ratings, such as Ultipor® N66, which incorporates a nylon 6,6 polymer membrane and is available from Pall Corporation). Preferably, the solution is filtered through more than one membrane (additional membrane filters are not illustrated).

After the membrane filtration, the filtrate is collected in a second reactor 4, wherein the product is precipitated by either concentrating or cooling the filtrate, or both. The precipitate is then transferred to filter drier 5, wherein impurities, including moisture, are removed. Preferably, the filtered product remains in the filter drier 5 to allow for further drying, such that a dry solid may be obtained. The drying may be accomplished by heating, pressure reduction, or both, provided that when the product is dried by heating, a subsequent cooling step is also performed. Following filtering/drying, the filter drier 5 containing the dried product is transferred into an LAF hood or glove box 6. Alternatively, the product may be filtered in a centrifuge drier and collected in sterile intermediate container 7. In the sterile environment of the LAF hood or glove box 6, the filter drier cover 5B is removed from the filter drier body SA, such that the sterile solid API may be discharged and collected in sterile intermediate container(s) 7.

The sterile solid API within the sterile intermediate container(s) 7 is then transferred to feeder 8, which controls the rate of feed of the API to a micronizer 9, preferably a jet mill, each of which is within LAF hood or glove box 11. Within micronizer 9, the API is pulverized to provide an API product of appropriate particle size. The product API from the micronizer 9 is weighed on scale 10, and then sampled and packaged in sterile container(s) 12. A similar apparatus is described in FIG. 2, where the filter (cartridge) 2 in FIG. 1 is replaced a series of filter (cartridges) for filter sterilizing the API and wherein not the filter drier 5 is transferred, but the API is discharged from the filter drier into an intermediate container 7 inside the sterile environment of a glove box 6. The API in the intermediate container 7 is then transferred to the sterile glove box 11 for micronization.

The apparatus, as described in FIGS. 1 and 2, is preferably sterilized before use by thermal treatment, i.e. the filter drier is submitted to a sterilization cycle with steam, and the filters and pipe lines are heated to about 122° C. under a steam flow. After use, the filters are washed with solvents suitable to remove residues of the remaining API. A suitable solvent is chosen in view of the API that is to be sterilized. In this respect the solvent for use in sterilizing the apparatus is the same solvent in which the API for preparing a packaged micronized sterile solid API is first dissolved. Preferably, suitable solvents are mainly polar solvents, such as alcohols, preferably C₁-C₄ alcohols, acetone, dimethylformamide (DMF), DMSO, Dioxane, Dimethyl acetamide, mixtures thereof with water, and water. A polar organic solvent refers to a solvent with a polarity index of higher than about 2.0.

Preferably, a solution of the API is prepared in a first reactor by dissolving the API in a suitable solvent. The choice of a suitable solvent to dissolve the API depends on the desired acceptable quality for the precipitate and/or crystal, such as starting particle size distribution (PSD) and polymorphic form. Examples of suitable solvents are methanol, acetone, dimethylformamide (DMF), DMSO, Dioxane, and Dimethyl acetamide. This step may be done under non-sterile conditions. Dissolution of the API may include a heating step. Preferably, the API is a high potency API selected from the group consisting of high potency API's that are used in compositions for inhalation and steroids. Examples of high potency API's that are used in inhalation compositions are Tiotropium and ciclesonide. Preferably, the steroid is a glucocorticosteroids such as Traimcinolone Acetonide, Medroxyprogesterone Acetate, Dexamethasone Base, Budesonide, and Methylprednisolone Acetate. More preferably the API is triamcinolone acetonide.

When the API is triamcinolone acetonide, the solvent is, preferably, a mixture of acetone and water. Preferably, dissolving triamcinolone acetonide in the mixture of acetone and water is done by heating to a temperature of about 35° C. to about 55° C., preferably about 40° C. to about 50° C. and more preferably about 45° C. to about 50° C.; wherein heating to a temperature below 60° C. is considered safe when dealing with steroids.

The solution may be filtered through one or more membranes, at least the last of which is a sterilizing filter. The filtrations are used to dispel microorganisms and other contaminating agents, and may be carried out under aseptic conditions such as for example in a glove box. The membranes may be of a cartridge type, made from a material that is compatible for fluids and solvents. Usually, three consecutive filtrations are done, wherein the first filtration is a pre-filtration used to protect subsequent membranes used for sterilization. In such later filtration the cartridge may be a sterilizing filter cartridge comprising a micron screen for sterilizing a solution, such as for example Ultipor N66 or a 0.22 micron sterilizing cartridge. Other sterilizing filtration cartridges or membranes comprise a membrane of polytetrafluoroethylene (PTFE), preferably Emflon, or comprising a membrane of polyvinylidenefluoride (PVDF), or a filtration grade nylon such as nylon 6.6. The second and third filter (filter cartridge) may be the same or a filter different from the first pre-filter. Preferably, the second and third filtrations are done subsequently. A preferred second cartridge is made from a polytetrafluoroethylene (PTFE) membrane, preferably Emflon, and a preferred third cartridge is made using a membrane of polyvinylidenefluoride (PVDF) or filtration grade nylon, such as nylon 6,6, preferably Novasip. When the API is triamcinolone acetonide, the filtration is done, preferably, while maintaining the temperature the same as in the dissolution step. However, when dissolution is obtained without heating, the filtration may also be conducted without heating.

The filtrate that passes the final or third membrane is collected in a second reactor, wherein precipitation of the product occurs. The precipitation may be induced by a step selected from the group consisting of: concentrating the filtrate, diluting the filtrate with an anti-solvent, cooling and a combination of these. In the process of the invention, precipitation may comprise crystallization of the solid sterile API. Such crystallization of the API may be carried out by adding an anti-solvent to the filtrate. The anti-solvent to induce precipitation and/or crystallization is preferably water. The anti-solvent may be added at a temperature of about 60° C. to about 90° C., preferably at about 75° C. to about 85° C., particularly where the API is triamcinolone acetonide. Concentrating the filtrate in the process of the present invention may be carried out by evaporation of the solvent. There where the filtrate is concentrated to precipitate or facilitate precipitation of the API the temperature of the dissolution step is preferably maintained. Preferably, a suspension is obtained when concentrating the filtrate and the suspension is cooled to a temperature of about 0° C. to about 20° C., preferably about 110° C. to about 20° C., more preferably about 15° C. to about 20° C. While cooling this suspension may be stirred. Cooling is carried out for a period sufficient to precipitate the API, preferably for a period of about 15 min to about 4 hours, more preferably for about 30 minutes to about 2 hours, most preferably for about 30 minutes.

Recovery of the precipitate preferably comprises filtering through a filter drier or a centrifuge drier, more preferably a filter drier. The filtered product may be maintained in the filter drier for further drying to obtain a dry solid. The drying may include a step selected from the group consisting of: heating, reducing the pressure, and combination of both. Preferably, heating is done to a temperature of about 30° C. to about 97° C. If the product is dried by heating, a subsequent cooling step is also performed. Preferably, cooling is done from a temperature of about 97° C. to about 20° C. The cooling step may be carried out over a period of time. When the API is triamcinolone acetonide, the drying process includes heating under reduced pressure. Preferably, heating is done to a temperature of about 85° C. to about 97° C., preferably 90° C. to about 97° C., more preferably 93° C. to about 97° C. Preferably, cooling is carried out to a temperature of about 15° C. to about 35° C., preferably to about 20° C. to about 30° C. This cooling step may be carried out for a period of about 6 hours to about 24 hours, preferably about 8 hours to about 18 hours, more preferably about 8 hours to about 12 hours.

After drying, the filter drier is discharged and the sterile solid API is packaged in sterile intermediate containers; wherein the unloading of the filter drier and material handling is done inside a sterile LAF hood or glove box. Preferably, the containers are sterilized by either gamma irradiation or by autoclaving.

The product obtained from the above process is then micronized in a sterilized micronizer contained in a sterile LAF hood or glove box. Preferably, the obtained product is fed into the micronizer from intermediate sterile containers. The micronization process can be done by any technique known to one skilled in the art, for example, a jet mill apparatus.

After the API is micronized it is weighed, sampled and packaged in sterile containers. Preferably, the containers are sterilized by either gamma irradiation or by autoclaving.

Having thus described the invention with reference to particular preferred embodiments and illustrative examples, those in the art can appreciate modifications to the invention as described and illustrated that do not depart from the spirit and scope of the invention as disclosed in the specification. Further, the disclosures of the references referred to in this patent application are incorporated herein by reference. The Examples are set forth to aid in understanding the invention but are not intended to, and should not be construed to limit its scope in any way.

### EXAMPLES

### Example 1

### Production of Sterile Solid Triamcinolone Acetonide

1 kg of Triamcinolone Acetonide was charged into a dissolution reactor, then 19.8 L of Acetone and 2.2 L of water were added. The suspension was heated to 45° C. to 50° C. until complete dissolution, and the solution was maintained at the temperature between 45° C. to 50° C. The solution was transferred through three membrane filers (sterilizing cartridge ultipor N66, filtering cartridge Emflon and filtering cartridge Novasip) into a second reactor, suitable for crystallization and precipitation. After the filtration the filters were washed with 4 L of Acetone and then with 0.44 of apirogen water. The filtered solution in the second reactor was evaporated under vacuum, maintaining the internal temperature around 50° C., until 3 L of residual volume remained. The suspension obtained in this way was cooled to 15° C. to 20° C., and stirred at this temperature for 30 minutes. Then, the suspension was filtrated in a filter dryer and solid washed with 6 L of apirogen water. Then the filter drier was kept under vacuum at 95±2° C. for almost 8 hours, followed by discharging the solid through a glove box and packaged into sterile containers, and if necessary transferred to a micronizer apparatus placed into a glove box.

Microbiological quality of the batch was verified by performing sterility test and bacterial endotoxins analysis on representative samples from dried and micronized triamcinolone acetonide batches and critically monitoring the production environment. The table below shows data supporting the sterility assurance of the batches produced. Each batch was sterile and with low content of bacterial endotoxins and the critical environment of production conforms to class A.

Results on Sterile Triamcinolone Acetonide

| | | Analysis | | | | |
|---|---|---|---|---|---|---|
| | | of the batches | | Environmental monitoring | | |
| | | | Bacterial | Air | Surface | |
| Step of | | Sterility | endotoxins | contamination | contamination | Personnel |
| production | Batches N^{o} | test | EU/mg | cfu/m³ | cfu/plate | cfu/Gloves |
| micronized | 6120SO90506 | sterile | <2.2 | <1 | <1 | <1 |
| dried | 30612091306 | sterile | <2.2 | <1 | <1 | <1 |
| | 30612091406 | sterile | <2.2 | <1 | <1 | <1 |
| | 30612091506 | sterile | <2.2 | <1 | <1 | <1 |
| | 30612091606 | sterile | <2.2 | <1 | <1 | <1 |
| | 30612091706 | sterile | <2.2 | <1 | <1 | <1 |
| | 30612091806 | sterile | <2.2 | <1 | <1 | <1 |
| micronized | 6120SO90107 | sterile | <2.2 | <1 | <1 | <1 |
| dried | 30612090107 | sterile | <2.2 | <1 | <1 | <1 |
| | 30612090207 | sterile | <2.2 | <1 | <1 | <1 |
| | 30612090307 | sterile | <2.2 | <1 | <1 | <1 |
| | 30612090407 | sterile | <2.2 | <1 | <1 | <1 |
| | 30612090507 | sterile | <2.2 | <1 | <1 | <1 |
| | 30612090707 | sterile | <2.2 | <1 | <1 | <1 |
| | 30612090807 | sterile | <2.2 | <1 | <1 | <1 |

### Example 2

### Production of Sterile Solid Triamcinolone Acetonide

29 L of apirogen water was charged into the dissolution reactor, transferred through a membrane filter (sterilizing cartridge ultipor Nylon66) into a second reactor, suitable for precipitation. The water was heated to 80±2° C. 0.5 kg of Triamcinolone Acetonide was charged into the dissolution reactor, then 2.6 L of DMF were added. The suspension was heated to 75±5° C. with stirring until complete dissolution, and the solution was maintained at the same temperature. The solution was transferred through three membrane filters into a second reactor, suitable for crystallization and precipitation. The filters were washed with 1 L of DMF and the suspension was maintained at 80±2° C. for not less then 1 hour with stirring. Then, the suspension was filtered in a filter dryer and the solid washed with twice with 10 L of apirogen preheated water (80±2° C.). Then the filter drier was kept under vacuum at 95±2° C. for 12-24 hours, followed by discharging the solid through a glove box and packaged into sterile containers, and if necessary transferred to a micronizer apparatus placed into a glove box. Yields are about 480 grams

Microbiological quality of the batch was verified performing sterility test and bacterial endotoxins analysis on representative samples from dried and micronized triamcinolone acetonide batches and critically monitoring the production environment. The table below shows data supporting sterility assurance of the batches produced. Each batch was sterile and with low content of bacterial endotoxins and the critical environment of production conforms to class A.

Results on Sterile Triamcinolone Acetonide

| | | Analysis | | | | |
|---|---|---|---|---|---|---|
| | | of the batches | | Environmental monitoring | | |
| | | | Bacterial | Air | Surface | |
| Step of | | Sterility | endotoxins | contamination | contamination | Personnel |
| production | Batches N^{o} | test | EU/mg | cfu/m³ | cfu/plate | cfu/Gloves |
| micronized | | sterile | <2.2 | <1 | <1 | <1 |
| dried | | sterile | <2.2 | <1 | <1 | <1 |
| | | sterile | <2.2 | <1 | <1 | <1 |
| | | sterile | <2.2 | <1 | <1 | <1 |
| | | sterile | <2.2 | <1 | <1 | <1 |
| | | sterile | <2.2 | <1 | <1 | <1 |
| | | sterile | <2.2 | <1 | <1 | <1 |

### Example 3

### Production of Sterile Solid Medroxyprogesterone Acetate

1 kg of Medroxyprogesterone Acetate was charged into the dissolution reactor, then 2.5 L of Dioxane were added. The suspension was heated to 80±5° C. with stirring until complete dissolution, and the solution was maintained at the same temperature. The solution was transferred through a membrane filter (sterilizing cartridge ultipor Nylon66) into a second reactor, suitable for crystallization and precipitation. The filters were washed with preheated Dioxane (0.3 L, 80±5° C.). 1.3 L of apirogen water was charged into the dissolution reactor and heated to 80±5° C., then transferred through three membrane filters into the second reactor, suitable for precipitation. After 10 minutes 4 L of apirogen water was charged into the dissolution reactor and heated to 80±5° C., then transferred again through three membrane filters into the second reactor. The suspension was maintained at 80±5° C. for not less then 1 hour with stirring. Then, the suspension was filtered in a filter dryer and the solid washed with twice with 1.5 L of apirogen preheated water (80±5° C.). Then the filter drier was kept under vacuum at 90±2° C. for 12-24 hours, followed by discharging the solid through a glove box and packaged into sterile containers, and if necessary transferred to a micronizer apparatus placed into a glove box. Yields are about 960 grams

### Example 4

### Production of Sterile Solid Medroxyprogesterone Acetate

1 kg of Medroxyprogesterone Acetate was charged into the dissolution reactor, then 3 L of DMA were added. The suspension was heated to 80±5° C. with stirring until complete dissolution, and the solution was maintained at the same temperature. The solution was transferred through a membrane filter (sterilizing cartridge ultipor Nylon66) into a second reactor, suitable for crystallization and precipitation. The filters were washed with preheated DMA (0.3 L, 80±5° C.). 1.2 L of apirogen water was charged into the dissolution reactor and heated to 80±5° C., then transferred through three membrane filters into the second reactor, suitable for precipitation. After 10 minutes 5 L of apirogen water was charged into the dissolution reactor and heated to 80±5° C., then transferred again through three membrane filters into the second reactor. The suspension was maintained at 80±5° C. for not less then 1 hour with stirring. Then, the suspension was filtered in a filter dryer and the solid washed with twice with 1.5 L of apirogen preheated water (80±5° C.). Then the filter drier was kept under vacuum at 90±2° C. for 12-24 hours, followed by discharging the solid through a glove box and packaged into sterile containers, and if necessary transferred to a micronizer apparatus placed into a glove box.

Yields are about 960 grams.

### Example 5

### Production of Sterile Solid Medroxyprogesterone Acetate

The same reagents, solvents, ratios and temperatures reported in the example 3 have been applied, but the Dioxane solution was filtered into the precipitation reactor already containing the apirogen water for the precipitation.

Yields obtained are the same of the example 3 previously reported

### Example 6

### Production of Sterile Solid Medroxyprogesterone Acetate

The same reagents, solvents, ratios and temperatures reported in the example 4 have been applied, but the DMA solution was filtered into the precipitation reactor already containing the apirogen water for the precipitation

Yields obtained are the same of the example 4 previously reported

What is claimed is:

1. A process to produce a micronized packaged sterile solid active pharmaceutical ingredient (API) in a laminar air flow (LAF) hood or glove box comprising the steps of a) providing a solution of the API, b) filtering the solution; c) precipitating and recovering the API from the solution; d) micronizing the API; and e) packing the API, wherein at least the steps d) and e) are carried out in a sterile LAF hood or glove box.

2. The process of claim 1, wherein steps c), d), and e) are carried out in a LAF hood or glove box.

3. The process of claim 1, wherein all steps with the exception of step a) are carried out in aseptic conditions.

4. The process of claim 3, wherein the aseptic conditions are inside a sterile LAF hood or glove box.

5. The process of claim 1, wherein the API is a high potency API selected from the group consisting of high potency API's that are used in inhalation compositions, high potency API's that are used in parenteral compositions, and steroids.

6. The process of claim 5, wherein the high potency API that is used in inhalation compositions is Tiotropium or ciclesonide.

7. The process of claim 5, wherein the API is a glucocorticosteroid.

8. The process of claim 7, wherein the glucocorticosteroid is selected from the group consisting of Traimcinolone Acetonide, Medroxyprogesterone Acetate, Dexamethasone Base, Budesonide, and Methylprednisolone Acetate.

9. The process of claim 1, wherein the solution of the API is prepared by dissolving the API in a solvent.

10. The process of claim 9, wherein the solvent is a polar solvent.

11. The process of claim 10, wherein the solvent is selected from the group consisting of alcohols, acetone, dimethylformamide (DMF), DMSO, Dioxane, Dimethyl acetamide, mixtures thereof with water, and water.

12. The process of claim 11, wherein the API is triamcinolone acetonide and the solvent is a mixture of acetone and water.

13. The process of claim 9, wherein the mixture of the API and the solvent are heated to dissolve the API in the solvent.

14. The process of claim 13, wherein the mixture of the API and the solvent are heated to a temperature of about 35° C. to about 55° C.

15. The process of claim 13, wherein the API is triamcinolone acetonide and is dissolved in a mixture of acetone and water by heating the mixture to a temperature of about 45° C. to about 50° C.

16. The process of claim 1, wherein filtering comprises filtration through one or more membranes, at least one of which is a sterilizing membrane.

17. The process of claim 16, wherein the filtration is carried out in a LAF hood or glove box.

18. The process of claim 16, wherein the membrane is selected from a polytetrafluorethylene (PTFE) membrane, a polyvinylidenefluoride (PVDF) membrane, and a nylon 6.6 membrane.

19. The process of claim 16, wherein the filtration comprises at least two consecutive filtrations.

20. The process of claim 19, wherein the filtration comprises three consecutive filtrations.

21. The process of claim 20, wherein the first filtration is a pre-filtration used for sterilization, the second filtration is through a polytetrafluorethylene (PTFE) membrane, and the third filtration is through a polyvinylidenefluoride (PVDF) or filtration grade nylon membrane.

22. The process of claim 16, wherein the filtration is carried out at the same temperature at which the solution of the API is obtained by dissolving the API in a solvent.

23. The process of claim 1, wherein precipitating the API is induced by a step selected from the group consisting of: concentrating the filtrate, adding an anti-solvent to the filtrate, cooling the filtrate, and a combination thereof.

24. The process of claim 23, wherein the concentrating step is carried out at the same temperature at which the filtering step is carried out.

25. The process of claim 23, wherein precipitating the API comprises concentrating the filtrate and cooling the concentrated filtrate to a temperature of about 0° C. to about 20° C.

26. The process of claim 25, wherein cooling is carried out for a period of about 15 min to about 4 hours.

27. The process of claim 23, wherein the anti-solvent is water.

28. The process of claim 27, wherein the API crystallizes from the filtrate.

29. The process of claim 28, wherein the API is triamcinolone acetonide and the anti-solvent water is added at a temperature of about 60° C. to about 90° C.

30. The process of claim 29, wherein the anti-solvent water is added at a temperature of about 75° C. to about 85° C.

31. The process of claim 1, wherein recovering the precipitated API comprises filtering through a filter drier or centrifuge drier.

32. The process of claim 31, wherein filtering is through a filter drier and further comprising drying the recovered API in the filter drier.

33. The process of claim 32, wherein drying comprises a step selected from the group consisting of: heating the recovered API, reducing the pressure in the filter drier, and a combination thereof.

34. The process of claim 33, wherein heating is to a temperature of about 30° C. to about 97° C.

35. The process of claim 34, further comprising cooling the dried API to a temperature of about 15° C. to about 35° C.

36. The process of claim 34, wherein the API is triamcinolone acetonide and heating is to a temperature of about 93° C. to about 97° C.

37. The process of claim 32, wherein packaging the recovered API comprises carrying out in a sterile LAF hood or glove box the steps of unloading the filter drier and packaging the sterile solid API in sterile containers.

38. The process of claim 1, wherein the process is carried out in an apparatus of the diagram in FIG. 1 or FIG. 2.

39. The process of claim 38, wherein the apparatus is first sterilized.
